(19) United States
(12) Patent Application Publication
(10) Pub. No.: US 2013/0012754 A1
(43) Pub. Date: Jan. 10, 2013

(54) MICROWAVE INDUCED PHOTODYNAMIC THERAPY

(75) Inventor: Yansong Gu, Bellevue, WA (US)

(73) Assignee: EMPIRE TECHNOLOGY DEVELOPMENT LLC

(21) Appl. No.: 13/320,731

(22) PCT Filed: Jul. 7, 2011
(86) PCT No.: PCT/US11/43148

§ 371 Date: Nov. 15, 2011

(51) Int. Cl. A61K 47/34; A61K 31/409; A61P 35/00; A61M 37/00

(52) U.S. Cl. 600/1; 514/772.7; 514/410

(57) ABSTRACT

A method of administering photodynamic therapy includes using a microwave frequency-sensitive light-emitting polymer as a light source to excite a administered photosensitizer to exert cytotoxicity in situ at a site containing undesired or diseased tissue.

### FIELD

The present technology relates to methods of photodynamic therapy and thermal hydrogel compositions for use therein.

### BACKGROUND

The following discussion of the background is merely provided to aid the reader in understanding the technology and is not admitted to describe or constitute prior art to the present application.

In photodynamic therapy (PDT), photosensitizing agents are provided to cells, and then light is used to activate the photosensitizing agents and to trigger the killing of diseased cells. Standard laser technology and light-emitting diode technology are approved for providing the light for treating diseases including tumors that are localized either on or under the skin, or on the lining of some internal organs such as the esophagus. However, PDT is less effective in treating large tumors and metastasis due to the inability of light to penetrate through more than one centimeter of tissue.

To circumvent the limitations of external light sources, implanting lighting devices at disease sites has been the focus of recent PDT research. Clinical trials have been conducted to research the applicability of “intratumoral placement of a nonlaser light device that activates talaporfin sodium,” an experimental photosensitizer (Lustig et al., A Multicenter Phase I Safety Study of Intratumoral Photoactivation of Talaporfin Sodium in Patients with Refractory Solid Tumors, Cancer, Vol. 98, No. 8, 2003), and which mount an optode on a flexible substrate and encapsulate it in a biocompatible epoxy for implantation (Margallo-Balbas et al., Telemetric light delivery and monitoring system for photodynamic therapy based on solid-state optodes Proc. SPIE 6852, 68520O (2008)). However, the approach presumably involving implanting new lighting devices requires the design and manufacture of an implant unit including a light source, a power supply, and a control panel, making it impractical for intratumoral implantation or for implantation in the variable area surrounding a tumor.

There is a need, therefore, for methods of intratumoral or intraperitoneal PDT that can overcome one or more of the existing limitations such as but not limited to tissue depth and implant size and shape.

### SUMMARY

In one aspect, a method of administering photodynamic therapy to tissue is provided, the method including administering a composition including a microwave frequency-sensitive light-emitting agent to the tissue; and irradiating the composition with a microwave frequency sufficient to activate the microwave frequency-sensitive light-emitting agent. In some embodiments, the method may further include the step of administering a photosensitizer to a patient in need of said therapy. In additional embodiments, the tissue is a disease site. Such tissues include, but are not limited to, solid tumors.

In some embodiments, the microwave frequency-sensitive light-emitting agent includes a diazoluminomelanin biopolymer. In some embodiments, the composition may further include a thermal hydrogel or reversible thermal hydrogel, which itself may optionally include a biodegradable aqueous copolymer, and wherein molecules of a chemotherapeutic agent or, alternatively, a photosensitizer, may be loaded into pores of the hydrogel and released over time. In other embodiments, the thermal hydrogel includes sodium bicarbonate, hydrogen peroxide, or an additional photosensitizer. In further embodiments, the biodegradable aqueous copolymer includes poly(N-isopropyl acrylamide), polyethylene glycol, methylcellulose or a hydroxyalkyl derivative thereof, polyoxyalkylene, an ionic polysaccharide, and/or a polymeric crosslinking agent. In some embodiments, the polymeric crosslinking agent is hydrophilic.

In some embodiments, the photosensitizer includes Porfimer sodium, Visudyne, Levulan, Foscan, Metvix, Hexvix, Laserphyrin, Antrin, Photochlor, Photosens, Photrex, aminolevulinic acid (ALA), silicon Phthalocyanine Pc, m-tetrahydroxyphenylchlorin (mTHPC), or mono-L-aspartyl chlorin e6 (NPe6).

In still other embodiments, the microwave frequency is applied via a radar transmitter operating at 1.23-2.5 GHz.

In a second aspect, a method of administering photodynamic therapy to a disease site is provided, the method including implanting a thermal hydrogel device including a microwave frequency-sensitive light-emitting agent at or near the disease site; and irradiating the device with a microwave frequency sufficient to activate the microwave frequency-sensitive light-emitting agent. In some embodiments, the method further includes the step of administering a photosensitizer to a patient in need of said therapy. In some embodiments, the thermal hydrogel device includes diazoluminomelanin in a thermal hydrogel; and a biocompatible adhesive coating; wherein the thermal hydrogel device is shaped to fit an anatomical space at or near the disease site. In other embodiments, the photosensitizer may be included in the thermal hydrogel. In another embodiment, the disease site is a solid tumor. In still other embodiments, the radio-microwave frequency is applied via a radar transmitter operating at 1.23-2.5 GHz.

In another aspect, a thermal hydrogel device is provided including a microwave-frequency sensitive light-emitting agent; a thermal hydrogel; and, in some embodiments, a biocompatible adhesive coating. In some embodiments, the thermal hydrogel device may further include a photosensitizer.

In yet another aspect, a composition is provided which includes a thermal hydrogel; wherein said thermal hydrogel includes diazoluminomelanin. The thermal hydrogel may, in some embodiments, further include a biodegradable aqueous copolymer, which may itself further include materials selected from poly(N-isopropyl acrylamide), polyethylene glycol, methylcellulose or a hydroxyalkyl derivative thereof, polyoxyalkylene, an ionic polysaccharide, and a polymeric crosslinking agent.

The foregoing summary is illustrative only and is not intended to be in any way limiting. In addition to the illustrative aspects, embodiments, and features described above, further aspects, embodiments, and features will become apparent by reference to the following drawings and the detailed description.

### DETAILED DESCRIPTION

The embodiments described in the detailed description and claims are illustrative only and are not intended to be limiting. Other embodiments may be used and changes may be made without departing from the scope of the subject matter presented here. It will be readily understood that the aspects of the present disclosure, as generally described herein can be arranged, substituted, combined, and designed in a wide variety of different configurations, all of which are explicitly contemplated and incorporated into this disclosure.

The definition of certain terms used herein are provided below. Unless defined otherwise, all technical and scientific terms used herein have the same meaning as commonly understood by one of ordinary skill in the art to which the present disclosure pertains.

As used herein, the term “hydrogel” refers to a three-dimensional polymeric structure that itself is insoluble in water but which is capable of absorbing and retaining large quantities of water to form a stable, often soft and pliable, but always to one degree or another shape-retentive, structure. The hydrogel may be in bulk form, that is, it is an amorphous mass of material with no discernable regular internal structure, or it may be a particulate hydrogel.

As used herein, a “thermal” hydrogel (or “thermogel”) is a type of temperature sensitive polymeric composition that transforms from a liquid form of lower viscosity to a semi-solid or solid form of higher viscosity, or vice versa, upon heating. The thermal hydrogels described herein may be reversible, wherein the change from liquid to semi-solid or solid form involves only physical forces such as hydrogen bonding, or irreversible, wherein the change from liquid to semi-solid or solid form involves chemical changes such as thermal-induced crosslinking.

As used herein, a “monomer” has the meaning understood by those skilled in the chemical art. That is, a monomer is a small chemical compound that is capable of forming a macromolecule of repeating units of itself, i.e., a homopolymer. Two or more different monomers may react to form a polymer in which each of the monomers is repeated numerous times, the polymer being referred to as a copolymer to reflect the fact that it is made up of more than one monomer.

As used herein, “disease site” refers to cells or tissue that have been affected by a disease generally treated with PDT or the area immediately surrounding the same. In some embodiments, the disease site is a solid tumor or the space immediately surrounding a solid tumor.

As used herein, “patient” refers to an animal (e.g., human or non-human) that has been diagnosed with a disease generally treated with PDT or as having an increased likelihood of developing a disease generally treated with PDT. Thus, the patient may be a human; a companion animal such as but not limited to a cat, dog, mouse, rat, guinea pig, or hamster; an amphibian such as but not limited to a frog, snake, lizard, or chameleon; a domesticated farm animal such as but not limited to a cow, pig, horse, chicken, duck, goose, or turkey; or a zoo animal among others.

The present technology relates generally to PDT using a radiomicrowave frequency-sensitive light emitting polymer and a photosensitizer. The methods include administering a composition that includes the radiomicrowave frequency-sensitive light emitting polymer to a site in a body, and exposing the composition to a microwave frequency that causes the polymer to emit light and activate the photosensitizer. The photosensitizer then produces singlet oxygen which triggers cell death in the surrounding tissue at the site. This allows administration of PDT to areas of the body which are impervious to light and allows for less invasive techniques to destroy tissue.

In some embodiments, the method includes administering a photosensitizer to a subject in need of photodynamic therapy. The administration of such a photosensitizer may be, for example, via parenteral routes directly to a particular site in the body, or parenterally, orally or rectally for general distribution throughout the patent. The method also includes administration of the microwave frequency-sensitive light-emitting agent either parenterally directly to the site, or parenterally, orally or rectally for general distribution throughout the patent. When both the photosensitizer and the light emitting agent are present at the desired site in the subject, the light emitting agent is then irradiated with a microwave frequency sufficient to activate the light-emitting agent. When the microwave frequency-sensitive light-emitting agent is activated, it emit lights that activates the photosensitizer thereby generating at least some singlet oxygen. The singlet oxygen may then act to destroy tissue located in and around the site. Such therapy may be used to destroy a wide variety of tissue, including, but not limited to, both malignant and benign tumor sites, other diseased, or unwanted, tissues. In some embodiments, the tissue is a solid tumor. Illustrative solid tumors may include, but are not limited to, prostate cancer, bladder cancer, lung cancer, and liver cancer. In yet other embodiments, the tissue may be infected by microorganisms, such as, but not limited to, a bacterial infection. In further embodiments, the tissue is adipose tissue. For example, the tissue may be diseased or physiologically abnormal adipose tissue, such as in patients with metabolic syndromes, such as obesity, or patients who receive adipose tissue transplantation for cosmetic purposes.

In one embodiment, both the light emitting agent and the photosensitizer are administered parenterally, directly to the desired location. In other embodiments, the light emitting agent is administered to the desired site parenterally, and the photosensitizer is administered parenterally, orally, or rectally to provide the photosensitizer to the desired site. Further, the photosensitizer and the light emitting agent may be administered concurrently as components of a hydrogel or thermogel. In either embodiment, when both the light emitting agent and the photosensitizer are present at the desired location, irradiation may be initiated.

As used herein, a photosensitizer is a compound that upon absorption of light, induces a chemical or physical alteration of another chemical entity. In some embodiments, the photosensitizers are based on a porphyrin structure, and are preferentially absorbed by dividing cells, such as cancer cells. The photosensitizers may be chemically synthesized or induced endogenously by an intermediate in heme synthesis, 5-aminolevulinic acid (5-ALA) or 5-ALA esters. The therapeutic effect imparted by the photosensitizer is based on the formation of reactive oxygen species (ROS) upon activation by light. Singlet oxygen is assumed to be the most important ROS for the therapeutic outcome (i.e. cytotoxicity). For a complete discussion, see Berg et al., Porphyrin-Related Photosensitizers For Cancer Imaging And Therapeutic Applications, J. Microsc., Vol. 218, May 2005, 133-147.

The selection of a photosensitizer will depend at least in part on the radio-microwave frequency, which determines the wavelength of the light-emitting agent. The wavelength, once determined, may then be paired with one or more suitable sensitizers that display desirable tissue distribution and/or uptake. Some suitable photosensitizers (and their corresponding wavelength for photoactivation) include, for example, porfimer sodium (Photofrin®, 630 nm), visudyne (693 nm), levulan (ALA or aminolevulinic acid, blue light), foscan (652 nm), metvix (630 nm), -laserphyrin (NPe6 or mono-L-aspartyl chlorine e6, 664 nm), photochlor, photosens (675 nm), photrex (verteporfin, 689 nm), silicon Phthalocyanine Pc (600-700 nm), m-tetrahydroxyphenylchlorin (mTHPC, 652 nm)), and azadipyrromethene (692 nm).

Photosensitizers may be administered orally, parenterally (e.g. intramuscular, intraperitoneal, intravenous, or subcutaneous injection), or by intrathecal or intracerebroventricular injection in an admixture with a pharmaceutically acceptable carrier adapted for the route of administration. In some embodiments, the photosensitizer may be administered concurrently with the radiomicrowave frequency-sensitive light emitting polymer. In other embodiments, the photosensitizer may be administered prior to the radiomicrowave frequency-sensitive light emitting polymer. For example, the photosensitizer may be administered as a bolus injection up to 2 days prior to the radiomicrowave frequency-sensitive light emitting polymer. The amount of sensitizer used may be based upon that used in other PDT procedures. In some embodiments, the photosensitizer is administered from about 0.1 mg/Kg to about 100 mg/kg of body weight of the subject receiving the photosensitizer. In one embodiment, the amount of photosensitizer is about 30 mg/kg of body weight.

As described herein, microwave frequency-sensitive light-emitting agents may be used to provide light. In some embodiments, the agent is the light-emitting polymer diazoluminomelanin (DALM; Formula I). DALM is a microwave-absorbing, biosynthesized polymer that has been shown to be chemiluminescent.

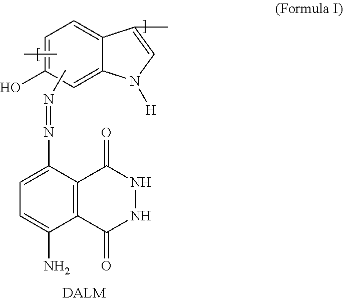

Although not necessary to obtain light emission from DALM, the emission may be enhanced when the DALM is in the presence of sodium bicarbonate and/or hydrogen peroxide. Accordingly, in some, embodiments, the compositions include an alkali metal bicarbonate and/or hydrogen peroxide. Suitable alkali metal bicarbonates include those of lithium, sodium, and potassium.

For application in PDT, the concentration of DALM polymers can be adjusted to maximize the efficiency for radio-microwave activation. For example, lower concentrations of the DALM will provide a lower amount of light emission upon microwave activation, and higher concentrations will provide higher amounts of light emission. Depending upon the tumor or location, the concentrations may be adjusted accordingly. The DALM polymers may be included in a hydrogel, thermal hydrogel, or similar biomaterial for purposes of administration and sequestration of the DALM near the desired site in the subject.

Hydrogels are generally understood to be lightly cross-linked networks of water soluble polymers. Hydrogels typically are capable of absorbing, but not dissolving in, water. Hydrogels find use in many applications due, in part, to their unique physical properties, including high porosity and the ability to absorb significant quantities of water. Non-limiting examples of hydrogels include poly(ethylene glycol) (PEG), poly(ethylene oxide) (PEO), poly(acrylic acid) (PAA), poly(methyacrylic acid) (PMAA), poly(2-hydroxyethyl methacrylate) (pHEMA), poly(vinyl alcohol) (PVA), poly(N-isopropylacrylamide) (PNIPAAM), poly(lactic acid) (PLA), poly(glycolic acid) (PGA), agarose, chitosan, and combinations thereof, including copolymers and blends thereof. In one embodiment, the hydrogel includes a co-polymer of the PNIPAAM with PVA or polyethylene glycol (PEG). Hydrogels may also include water soluble polymers that are adapted to form physical cross-links with other molecules, including other hydrogel precursor monomers. These cross-linking agents may be based on physicochemical interactions such as hydrophobic interactions, charge condensation, hydrogen bonding, stereocomplexation, or supramolecular chemistry. Additionally, the crosslinking agent may be hydrophilic.

According to one embodiment, the hydrogel may be a thermal hydrogel. For example, the hydrogel may be a relatively non-viscous liquid at a higher temperature, and a relatively viscous gel at lower temperature. Alternatively, the hydrogel may be relatively non-viscous at lower temperature and become viscous or solid at higher temperature. Such relative temperatures may be separated by only a few degrees. For example, the hydrogel may be a non-viscous liquid at temperatures slightly above physiological temperatures, but a viscous gel at physiological temperatures such that at the higher temperature the hydrogel is amenable to injection to a disease site. However, once administered the hydrogel becomes a viscous gel to hold the hydrogel at the disease/injection site. Alternatively, the hydrogel may be a non-viscous liquid at temperatures slightly below physiological temperatures, but a viscous gel at physiological temperatures such that at the lower temperature the hydrogel is amenable to injection to a disease site. However, once administered the hydrogel becomes a viscous gel to hold the hydrogel at the disease/injection site.

The hydrogels may be bulk hydrogels or particulate hydrogels. Bulk hydrogels have slow swelling rates due to the large internal volume relative to the surface area through which water must be absorbed. Furthermore, a substance dissolved or suspended in the absorbed water will diffuse out of the gel at a rate that depends on the distance it must travel to reach the outer surface of the gel. This situation can be ameliorated to some extent by using particulate hydrogels. If each particle is sufficiently small, substances dispersed in the particles will diffuse to the surface and be released at a predictable rate.

Particulate hydrogels can be formed by a number of procedures such as direct or inverse emulsion polymerization (Macromolecules 33:2370, (2000)) or they can be created from bulk hydrogels by drying the gel and then grinding the resulting xerogel to small particles of a desired size. The particles can then be re-solvated to form particulate gels. Particles having sizes in the micro (10⁻⁶ meters (m)) to nano (10⁻⁹ m)) diameter range can be produced by this means. Molecules of a substance occluded by particles in these size ranges will all have about the same distance to travel to reach the outer surface of the particle and will exhibit in some cases near zero-order release kinetics.

The hydrogels provide several functions in the compositions. One such function is to hold the radiomicrowave frequency-sensitive light emitting polymer at the desired location in the body. For example, the hydrogel, when administered to the body, gels and either prevents or reduces the diffusion of the light emitting polymer from the site of administration. Another function, where both the radiomicrowave frequency-sensitive light emitting polymer and the photosensitizer are administered in a single composition, is to hold both materials in close proximity to one another and prevent, or reduce their diffusion from the site of administration. Another function maybe to include another chemotherapeutic agent, at or near the site of administration with the PDT materials, and to slowly release the chemotherapeutic agent from the hydrogel over time. For example, once the PDT is completed, the chemotherapeutic agent may diffuse from the hydrogel to continue treatment of the disease.

The hydrogel may be administered parenterally. Formulations for parenteral administration include sterile aqueous or non-aqueous solutions, suspensions, or emulsions. Examples of suitable vehicles include propylene glycol, polyethylene glycol, vegetable oils, gelatin, hydrogenated naphthalenes, and injectable organic esters, such as ethyl oleate. Such formulations may also contain adjuvants, such as preserving, wetting, emulsifying, and dispersing agents. Biocompatible, biodegradable lactide polymer, lactide/glycolide copolymer, or polyoxyethylene-polyoxypropylene copolymers may be used to control the release of the compounds. Other potentially useful parenteral delivery systems for the polymers of the invention include ethylene-vinyl acetate copolymer particles, osmotic pumps, implantable infusion systems, and liposomes.

Further, the hydrogel may be fashioned into a device which may be implanted at or near the disease site. The device may include a microwave frequency sensitive light-emitting agent such as DALM, a hydrogel, a photosensitizer, or a biocompatible adhesive coating. In some embodiments, the hydrogel is shaped to fit the anatomical space at or near the disease site. For example, through imaging mapping of the disease site, a hydrogel containing the light emitting agent may be formed to fit around the disease site. Such a device would likely be implanted surgically. Accordingly, in another aspect, a thermal hydrogel device is provided which includes a microwave-frequency sensitive light-emitting agent; and a thermal hydrogel. In this aspect, a thermal hydrogel may be injected or implanted in liquid or gel form which will fill an existing anatomical space and solidify once injected or implanted. If the thermal hydrogel is a liquid or gel below body temperature, it may solidify upon injection or implantation, due to the comparative increase in temperature in the body. If the thermal hydrogel is a liquid or gel above body temperature, it may solidify upon injection or implantation, due to the comparative decrease in temperature in the body.

In one such embodiment, the hydrogel device has a layered structure, where the photosensitizer is in an inner layer and the microwave frequency sensitive, light-emitting agent is in an outer layer. This will allow the cell death to occur in a more targeted area, because only the light that is directed toward the disease site will have the effect of causing cell death. For example, a hydrogel containing the photosensitizer is placed or injected around a diseased site or around unwanted tissue. A hydrogel containing the light-emitting agent is then place or injected around the hydrogel containing the photosensitizer. When the light-emitting agent is activated by microwave frequency to emit light, any light going into the surrounding normal, or non-targeting tissue is merely harmlessly absorbed, whereas the light going into the photosensitizer causes the generation of singlet oxygen which may then cause cell death in tissue which is surrounded by the layered structure.

In another aspect, a method of administering photodynamic therapy to a disease site via a device is provided. Such methods include implanting one or more of the above devices in or near a disease site in a subject, the device including a microwave frequency-sensitive light-emitting agent at or near the disease site; and irradiating the device with a microwave frequency sufficient to activate the microwave frequency-sensitive light-emitting agent to emit light and activate a photosensitizer to generate singlet oxygen to destroy tissue. According to one embodiment, the method also includes administering the photosensitizer to a patient in need of said therapy. In one embodiment, the microwave frequency-sensitive light-emitting agent includes a diazoluminomelanin biopolymer.

The above compositions, hydrogels, or devices may be irradiated with a microwave frequency sufficient to activate the microwave frequency-sensitive light-emitting agent. A transmitter, operating at from about 1.25 to about 2.5 GHz provides pulses of 5.73+/−0.09 microwaves in duration at 10.00 pulses with 2.07+/−0.08 MW forward power activates the photosensitizer, which produces singlet oxygen molecules that damage and kill diseased cells at the site of hydrogel treatment. In a preferred embodiment, the microwave frequency is about 2450 MHz. The DALM will emit light of various wavelengths depending upon the wavelength of the applied microwaves.

Generally, when administered to a patient, the timing of the PDT will depend on the nature of the light-emitting agent, and can readily be determined by one skilled in the art. Each agent may be administered once or repeatedly over a period of time (e.g., including for the entire lifetime of the patient).

The present technology, thus generally described, will be understood more readily by reference to the following examples, which are provided by way of illustration and are not intended to be limiting.

### EXAMPLE

The methods and systems disclosed herein are further illustrated by the following examples, which should not be construed as limiting in any way.

### Example 1

Administration of PDT using a DALM hydrogel as the light-emitting agent. Patients with a biopsy-confirmed, non-resectable persistent thyroid cancer are to be given intravenous Photofrin® at a dosage level of between 0.1 mg/kg to 100 mg/kg of body weight as a bolus injection over 3-5 minutes. Approximately 48 hours later, a thermal hydrogel of DALM, PEG, and a hydrophilic crosslinking agent, as well as sodium bicarbonate, hydrogen peroxide, and additional Photofrin®, is to be administered intratumorally. A radar transmitter, operating at 2450 MHz provides pulses of 5.73 pulses per microsecond microwaves in duration at 10.00 pulses per second with 2.07 MW forward power, is applied.

### EQUIVALENTS

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure. Applicants reserve the right to physically incorporate into this application any and all materials and information from any such articles, patents, patent applications, or other physical and electronic documents.

The embodiments, illustratively described herein, may suitably be practiced in the absence of any element or elements, limitation or limitations, not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like, includes the number recited and refers to ranges which can be subsequently broken down into subranges as discussed above. Finally, as will be understood by one skilled in the art, a range includes each individual member.

Other embodiments are set forth in the following claims.

1. A method of administering photodynamic therapy to a disease site comprising:
administering a composition comprising a microwave frequency-sensitive light-emitting agent to the disease site; and
irradiating the composition with a microwave frequency sufficient to activate the microwave frequency-sensitive light-emitting agent to emit light and activate a photosensitizer;
wherein the microwave frequency-sensitive light-emitting agent comprises a diazoluminomelanin biopolymer.

2. The method of claim 1, further comprising the step of administering the photosensitizer to a patient in need of said therapy.

3. (canceled)

4. The method of claim 2, wherein the photosensitizer and the composition are administered as a single formulation.

5. The method of claim 1, wherein the composition further comprises a thermal hydrogel.

6. The method of claim 5, wherein molecules of a chemotherapeutic agent are loaded into pores of the hydrogel and released over time.

7. The method of claim 5, wherein the thermal hydrogel further comprises a biodegradable aqueous polymer.

8. The method of claim 5, wherein the thermal hydrogel further comprises a photosensitizer.

9. The method of claim 7, wherein the biodegradable aqueous polymer comprises a material selected from the group consisting of: poly(N-isopropyl acrylamide), polyethylene glycol, methylcellulose or a hydroxyalkyl derivative thereof, polyoxyalkylene, polylactic acid, poly glycolic acid, an ionic polysaccharide, and blends or copolymers thereof.

10. The method of claim 7, wherein the biodegradable aqueous polymer further comprises a cross-linking agent.

11. The method of claim 1, wherein the photosensitizer comprises Porfimer sodium, Visudyne, Levulan, Foscan, Metvix, Hexvix, Laserphyrin, Antrin, Photochlor, Photosens, Photrex, aminolevulinic acid (ALA), silicon Phthalocyanine Pc, m-tetrahydroxyphenylchlorin (mTHPC), or mono-L-aspartyl chlorine e6 (NPe6).

12. The method of claim 1, wherein the thermal hydrogel further comprises sodium bicarbonate, hydrogen peroxide, or an additional photosensitizer.

13. The method of claim 1, wherein the microwave frequency is applied via a radar transmitter operating at 1.23-2.5 GHz.

14. The method of claim 2, wherein the photosensitizer is administered as a bolus injection up to 48 hours prior to the administration of the microwave frequency-sensitive light-emitting agent.

15. A method of administering photodynamic therapy to a disease site comprising:
implanting a device comprising a microwave frequency-sensitive light-emitting agent at, or near, the disease site; and
irradiating the device with a microwave frequency sufficient to activate the microwave frequency-sensitive light-emitting agent
wherein the microwave frequency-sensitive light-emitting agent comprises a diazoluminomelanin biopolymer.

16. The method of claim 15, further comprising the step of administering a photosensitizer to a patient in need of said therapy.

17. (canceled)

18. The method of claim 15, wherein the device is shaped to fit an anatomical space at or near the disease site.

19. A thermal hydrogel device comprising:
a microwave-frequency sensitive light-emitting agent; and
a thermal hydrogel.

20. The device of claim 19, further comprising a photosensitizer.
